(19) United States
(12) Patent Application Publication
(10) Pub. No.: US 2016/0297820 A1
(43) Pub. Date: Oct. 13, 2016

(54) DIMERIZER COMPOUND

(71) Applicant: AGENCY FOR SCIENCE, TECHNOLOGY AND RESEARCH, Singapore (SG)

(72) Inventors: Yiqi SEOW, Singapore (SG); Fong Tian WONG, Singapore (SG); Yin Nah TEO, Singapore (SG)

(21) Appl. No.: 14/917,935

(22) PCT Filed: Sep. 9, 2014
(86) PCT No.: PCT/SG2014/000424

§ 371 Date: Mar. 9, 2016

(30) Foreign Application Priority Data
Sep. 9, 2013 (SG) 2013067855

(51) Int. Cl. C07D 473/18

(52) U.S. Cl. CPC C07D 473/18

(57) ABSTRACT

There is provided a compound comprising at least two ligands that are individually coupled to a linker, wherein each ligand is independently selected from the group consisting of a substituted benzylguanine derivative, a substituted benzylcytosine derivative, a haloalkyl moiety, a drug, a peptide, a hormone, an inorganic compound and a protein. The compound may be part of an oligomer. The compound may be employed in a method of dimerizing a pair of proteins, which may alter a biological function in a cell. There is also provided a method of forming the compound.

### TECHNICAL FIELD

The present invention generally relates to a compound comprising at least two ligands that are individually coupled to a linker. The present invention also relates to an oligomer comprising the above compound coupled to respective cognate proteins.

### BACKGROUND

The ability to selectively dimerize and dissociate protein pairs via small ligands is beneficial in a number of biological processes. Dimerization and oligomerization can be induced by small molecule ligands which are comprised of two or more protein-specific ligand linked covalently together. Such dimerization can be used in research and manufacturing purposes as switches for a number of applications, including studying functional effects of protein-protein interaction, switching gene expression on/off and labeling proteins. For example, FKBP domains are fused to ErbB family of receptor tyrosine kinase and small molecular dimerization ligands are added to create homo- and hetero-dimers of these kinases independently of its natural endogenous ligands—growth factors.

However, the ligands commonly used for dimerization are not modular and not easily reversible. The lack of modularity means it is not easy to chemically derive novel dimerizers based on linking individual ligands covalently together. For example, the FKBP ligand had to be engineered extensively to develop novel heterodimerizers. Furthermore, the typical means of reversing dimerization in cells is to remove the dimerizer from the medium, which would reverse the dimerization events primarily via protein turnover.

Protein dimerization of O⁶-alkylguanine-DNA alkyltransferase-derived, O⁶-alkylguanine-DNA analog-binding domains (AGT) has been extensively studied and usually involves the use of covalent interaction between the proteins and a dimerizer. As the interaction is covalent, it is intrinsically not reversible.

There is a need to provide a compound dimerizer that overcomes, or at least ameliorates, one or more of the disadvantages described above.

### SUMMARY

According to a first aspect, there is provided a compound comprising at least two ligands that are individually coupled to a linker, wherein each ligand is independently selected from the group consisting of a substituted benzylguanine derivative, a substituted benzylcytosine derivative, a haloalkyl moiety, a drug, a peptide, a hormone, an inorganic compound and a protein.

Advantageously, the compound may be used to link (or dimerize) cognate binding protein partners of the individual ligands together for the purpose of effecting a biological function or inhibiting a biological function through dimerization. Advantageously, the compound may link cognate binding proteins together that do not usually interact naturally.

Advantageously, the above biological function can be terminated by reversing dimerization of the cognate binding proteins, when the linker is cleaved. The linker may be cleaved in conditions that are not naturally occurring in vivo in a cell when present in a biological organism and may be cleaved in conditions that are suitable for cellular survival in vitro. Hence, the linker may be non-toxic and may not naturally degrade within a cell.

The compound may be also termed as a dimerizer compound.

The compound may be synthesized in a modular manner in which the use of a modular backbone can be universally applied to, various types of ligand pairs. Each component of the compound (such as the ligand(s) or linker) can be treated as a modular unit, which can be synthesized with another modular unit to create the dimerizer compound with desired and specific function(s). Each modular unit can be interchangeable so as to elicit different effects or have different functions. As the method of assembling the various modular units is similar, this may allow a user to assemble new dimerizers for dimerizing specific proteins with the same methodology easily and conveniently.

According to a second aspect, there is provided an oligomer comprising a pair of ligands that are coupled to respective proteins to form ligand/protein pairs, wherein each ligand/protein pair is individually coupled to a linker, and wherein each ligand is independently selected from the group consisting of a substituted benzylguanine derivative, a substituted benzylcytosine derivative, a haloalkyl moiety, a drug, a peptide, a hormone, an inorganic compound and a further protein.

According to a third aspect, there is provided a method of dimerizing a pair of proteins comprising the step of incubating the pair of proteins with a compound comprising at least two ligands that are individually coupled to a linker, wherein each ligand is independently selected from the group consisting of a substituted benzylguanine derivative, a substituted benzylcytosine derivative, a haloalkyl moiety, a drug, a hormone, an inorganic compound and a further protein, said ligands being respective substrates for the proteins of the protein pair.

According to a fourth aspect, there is provided a method of altering a biological function in a cell, comprising the step of forming a dimer by incubating a pair of proteins with a compound comprising at least two ligands that are individually coupled to a linker comprising a cleavable moiety, wherein each ligand is independently selected from the group consisting of a substituted benzylguanine derivative, a substituted benzylcytosine derivative, a haloalkyl moiety, a drug, a hormone, an inorganic compound and a further protein, said ligands being respective substrates for the proteins of the protein pair.

According to a fifth aspect, there is provided a method of forming a compound as disclosed above, comprising the step of reacting a first ligand with a second ligand, wherein each ligand is independently selected from the group consisting of a substituted benzylguanine derivative, a substituted benzylcytosine derivative, a haloalkyl moiety, a drug, a hormone, an inorganic compound and a protein.

### Definitions

The following words and terms used herein shall have the meaning indicated:

The term ‘dimerizer’ is to be interpreted broadly to include a compound that is capable of causing or facilitating the formation of a dimer.

The term “alkyl” is to be interpreted broadly to include monovalent (“alkyl”) and divalent (“alkylene”) straight chain or branched chain saturated aliphatic groups having from 1 to 10 carbon atoms, eg, 1, 2, 3, 4, 5, 6, 7, 8, 9, or 10 carbon atoms. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like.

The term “amino” is to be interpreted broadly to include groups of the form —NRₐR_{b} wherein Rₐ and R_{b} are individually selected from the group including but not limited to hydrogen, optionally substituted alkyl, optionally substituted alkenyl, optionally substituted alkynyl, and optionally substituted aryl groups.

The term “acyl” is to be interpreted broadly to include groups of the form RCO— where R represents an alkyl group that is attached to the CO group with a single bond.

The term “alkenyl” is to be interpreted broadly to include monovalent (“alkenyl”) and divalent (“alkenylene”) straight or branched chain unsaturated aliphatic hydrocarbon groups having from 2 to 10 carbon atoms, eg, 2, 3, 4, 5, 6, 7, 8, 9, or 10 carbon atoms and having at least one double bond, of either E, Z, cis or trans stereochemistry where applicable, anywhere in the alkyl chain. Examples of alkenyl groups include but are not limited to ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like.

The term “alkynyl” is to be interpreted broadly to include monovalent (“alkynyl”) and divalent (“alkynylene”) straight or branched chain unsaturated aliphatic hydrocarbon groups having from 2 to 10 carbon atoms and having at least one triple bond anywhere in the carbon chain. Examples of alkynyl groups include but are not limited to ethynyl, 1-propynyl, 1-butynyl, 2-butynyl, 1-methyl-2-butynyl, 3-methyl-1-butynyl, 1-pentynyl, 1-hexynyl, methylpentynyl, 1-heptynyl, 2-heptynyl, 1-octynyl, 2-octynyl, 1-nonyl, 1-decynyl, and the like.

The term “aryl” is to be interpreted broadly to include monovalent (“aryl”) and divalent (“arylene”) single, polynuclear, conjugated and fused residues of aromatic hydrocarbons having from 6 to 10 carbon atoms. Examples of such groups include phenyl, biphenyl, naphthyl, phenanthrenyl, and the like.

The term “optionally substituted” as used herein means the group to which this term refers may be unsubstituted, or may be substituted with one or more groups independently selected from alkyl, alkenyl, alkynyl, thioalkyl, cycloalkyl, cycloalkenyl, heterocycloalkyl, halo, carboxyl, haloalkyl, haloalkynyl, hydroxyl, alkoxy, thioalkoxy, alkenyloxy, haloalkoxy, haloalkenyloxy, nitro, amino, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, alkylheteroaryl, cyano, cyanate, isocyanate, —C(O)NH(alkyl), and —C(O)N(alkyl)₂.

All isomeric forms of the compounds disclosed herein are included within the scope of the present invention, including all diastereomeric isomers, racemates and enantiomers. This includes, for example, E, Z, cis, trans, (R), (S), (L), (D), (+), and/or (−) forms of the compounds, as appropriate in each case.

The term “substituted” is intended to indicate that one or more (e.g., 1, 2, 3, 4, or 5; in some embodiments 1, 2, or 3; and in other embodiments 1 or 2) hydrogen atoms on the group indicated in the expression using “substituted” is replaced with a selection from the indicated organic or inorganic group(s), or with a suitable organic or inorganic group known to those of skill in the art, provided that the indicated atom's normal valency is not exceeded, and that the substitution results in a stable compound. Suitable indicated organic or inorganic groups include, e.g., alkyl, alkenyl, alkynyl, alkoxy, halo, haloalkyl, hydroxy, hydroxyalkyl, aryl, heteroaryl, heterocycle, cycloalkyl, alkanoyl, alkoxycarbonyl, amino, alkylamino, dialkylamino, trifluoromethylthio, difluoromethyl, acylamino, nitro, trifluoromethyl, trifluoromethoxy, carboxy, carboxyalkyl, keto, thioxo, alkylthio, alkylsulfinyl, alkylsulfonyl, alkylsilyl, and cyano. Additionally, the suitable indicated groups can include, e.g., —X, —R, —O—, —OR, —SR, —S—, —NR 2, —NR 3, ═NR, —CX 3, —CN, —OCN, —SCN, —N═C═O, —NCS, —NO, —NO 2, ═N 2, —N 3, NC(═O)R, —C(═O)R, —C(═O)NRR —S(═O) 2 O—, —S(═O) 2 OH, —S(═O) 2 R, —OS(═O) 2 OR, —S(═O) 2 NR, —S(═O)R, —OP(═O)O 2 RR, —P(═O)O 2 RR—P(═O)(O—) 2, —P(═O) (OH) 2, —C(═O)R, —C(═O)X, —C(S)R, —C(O)OR, —C(O)O—, —C(S)OR, —C(O)SR, —C(S)SR, —C(O)NRR, —C(S)NRR, —C(NR)NRR, where each X is independently a halogen (or “halo” group): F, Cl, Br, or I; and each R is independently H, alkyl, aryl, heterocycle, protecting group or prodrug moiety. As would be readily understood by one skilled in the art, when a substituent is keto (i.e., ═O) or thioxo (i.e., ═S), or the like, then two hydrogen atoms on the substituted atom are replaced.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Exemplary, non-limiting embodiments of a compound comprising at least two ligands that are individually coupled to a linker will now be disclosed. Each of the ligand may be independently selected from the group consisting of a substituted benzylguanine derivative, a substituted benzylcytosine derivative, a haloalkyl moiety, a drug, a peptide, a hormone, an inorganic compound and a protein.

The ligand may be a substituted benzylguanine derivative. The substituted benzylguanine derivative may have the following formula (I):

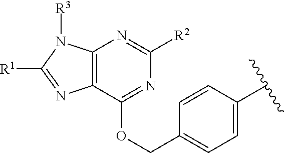

### (I)

wherein

R¹ is selected from hydrogen or alkyl;

R² is selected from amino, hydroxyl, alkylamino, dialkylamino or acylamino;

R³ is selected from hydrogen, aminoalkyl, alkyl or dialkylamino; and

[symbol] denotes the point of attachment to the linker.

In formula (I), R¹ and R³ may both be hydrogen and R² may be amino.

The alkyl, alkylamino, dialkylamino or acylamino groups of formula (I) may independently contain 1 to 10 carbon atoms.

The ligand may be a substituted benzylcytosine derivative. The substituted benzylcytosine derivative may have the following formula (II):

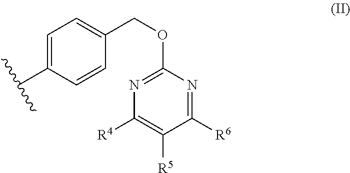

wherein

R⁴ is selected from amino, hydroxyl, alkylamino, dialkylamino or acylamino;

R⁵ is selected from hydrogen, aminoalkyl, alkyl or dialkylamino;

R⁶ is selected from hydrogen, aminoalkyl, alkyl or dialkylamino; and

[symbol] denotes the point of attachment to the linker.

In formula (II), R⁴ may be amino while R⁵ and R⁶ may both be hydrogen.

The alkyl, alkylamino, dialkylamino or acylamino groups of formula (II) may independently contain 1 to 10 carbon atoms.

The ligand may be a haloalkyl moiety, in which the alkyl group has 1 to 10 carbon atoms. The halo group may be selected from fluorine, chlorine, bromine, or iodine.

The ligand may be a drug. The drug may be selected from the group consisting of rapamycin, doxycycline and tetracycline.

The ligand may be a protein. The protein may be a peptide tag selected from the group consisting of a FLAG-tag, an AviTag, a calmodulin-tag, a HA-tag, a His-tag, a Myc-tag, a S-tag, a SBP-tag, a softag 1, a softag3, a V5 tag, a Xpress tag, an isopeptad, a SpyTag, glutathione-S-transferase-tag, green fluorescent protein-tag, maltose binding protein-tag, biotin carboxyl carrier protein-tag, Nus-tag, strep-tag, thioredoxin-tag, TC tag and Ty tag.

The ligand may be an inorganic compound such as nickel.

Each ligand may be different from each other or may be the same as each other. In an exemplary compound, one of the ligands may be a substituted benzylguanine derivative while the other ligand may be a substituted benzylcytosine derivative. In another exemplary compound, both ligands may be a substituted benzylguanine derivative. In yet another exemplary compound, both ligands may be a substituted benzylcytosine derivative.

The linker in the compound may be used to couple both of the ligands together. The linker may be chemically stable and may not interact with the cognate protein partners.

The linker may comprise a water-soluble polymeric moiety. Hence, the linker may increase the solubility of the compound in an appropriate solvent or allow the compound to be soluble in an aqueous environment (such as in a cell which is predominantly water). The water-soluble polymeric moiety may comprises monomers selected from the group consisting of alkylene glycols, alkylene pyrrolidones, alkylene alcohols, carboxylic acids, alkylene amides, alkyl acetates, hydroxyalkyls, oxazolines, phosphates, phosphazenes, saccharides, peptides, and combinations thereof. The water-soluble polymeric moiety may be selected from the group consisting of polyethylene glycol, polyvinyl pyrrolidone, polyvinyl alcohol, polyacrylic acid, polyacrylamides, N-(2-hydroxypropyl)methacrylamide, divinyl ether-maleic anhydride, polyoxazoline, polyphosphate, polyphosphazne, xanthan gum, pectin, chitin, chitosan, dextran, carrageenan, guar gum, hydroxypropylmethyl cellulose, hydroxypropyl cellulose, hydroxyethyl cellulose, sodium carboxy methyl cellulose, hyaluronic acid, albumin, starch and copolymers thereof.

The linker may further comprise one or more cleavable moiety. By having a cleavable moiety in the linker (and thereby in the compound), this may ensure that dimerization of the cognate protein partners may be controlled such that the dimerization may be terminated when the linker is cleaved. This may enable the duration or extent of a biological function (which occurs or is inhibited by the dimerization of the cognate protein partners) to be controlled in a simple manner by subjecting the compound to conditions that cause cleavage of the linker.

The cleavable moiety of the ligand may be cleaved by at least one of enzyme, basic reagent, reducing agent, photo-irradiation, acidic reagent and oxidizing agent.

Where the cleavable moiety is cleaved by an enzyme, the enzyme may be selected from the group consisting of trypsin, thrombin, cathepsin B, cathespin D, cathepsin K, caspase 1, matrix matelloproteinase, phosphodiesterase, phospholipidase, esterase and beta-galactosidase.

Where the cleavable moiety is cleaved in alkaline conditions (conditions that are created by adding a basic reagent), the cleavable moiety may be selected from the group consisting of dialkyl dialkoxysilane, cyanoethyl group, sulfone, ethylene, glycolyl disuccinate, 2-N-acyl nitrobenzenesulfonamide, alpha-thiophenylester, unsaturated vinyl sulfide, sulfonamide after activation, malondialdehyde-indole derivative, levulinoyl ester, hydrazine, acylhydrazone and alkyl thioester.

Where the cleavable moiety is cleaved by a reducing agent, the cleavable moiety may be at least one of a disulfide-containing moiety and an azo compound. Where the cleavable moiety is a disulfide-containing moiety, the reducing agent may be, but not limited to, dithiothreitol (DTT), beta-mercaptoethanol, or tris(2-carboxyethyl)phosphine (TCEP). Where the cleavable moiety is an azo compound, the reducing agent may be, but not limited to, sodium dithionite, DTT or TCEP.

Where the cleavable moiety is cleaved by photo-irradiation, the cleavable moiety may be selected from the group consisting of 2-nitrobenzyl derivative, phenacyl ester, 8-quinolinyl benzenesulfonate, coumarin, phosphotriester, bis-arylhydrazone and bimane-bisthiopropionic acid. The cleavable moiety may be selected from the group consisting of

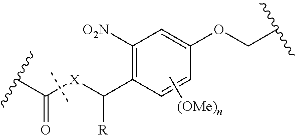

(wherein X is —NH— or —O— and R is methyl or hydrogen);

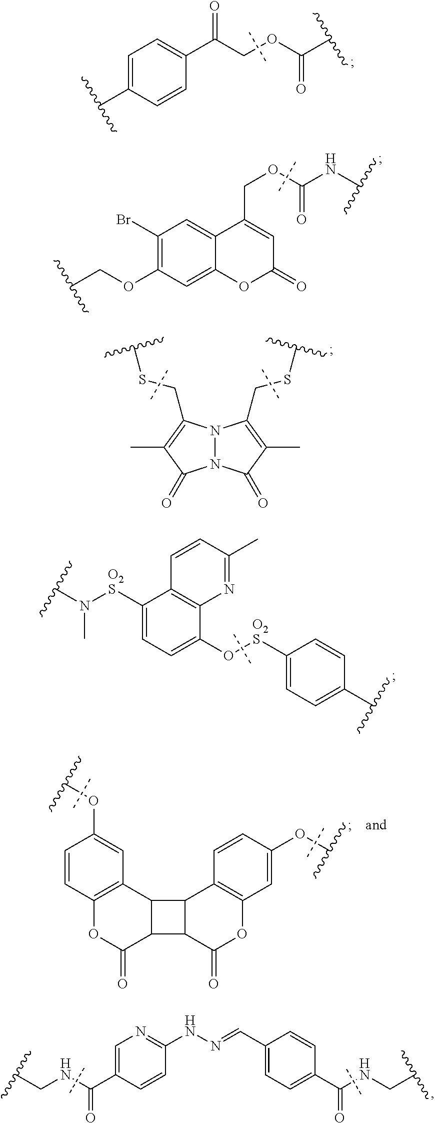

wherein the dashed lines ( - - - ) indicate the position of photo-cleavage. The photo-irradiation may be carried out using a UV light source that emits electromagnetic radiation with a wavelength in the range of about 10 nm to about 400 nm, about 250 nm to about 400 nm, about 300 nm to about 400 nm, about 350 nm to about 400 nm, about 280 nm to about 366 nm, about 300 nm to about 330 nm, about 300 nm to about 365 nm, or about 360 nm to about 370 nm. The wavelength of the UV light emitted may be about 365 nm. The photo-irradiation may be applied for any period of time, for example, about 5 to about 15 minutes, 5 minutes or 10 minutes. Photocleavable moieties may be chemically inert to changes in pH.

Where the cleavable moiety is cleaved in acidic conditions (conditions that are created by adding an acidic reagent), the cleavable moiety may be selected from the group consisting of tert-butyloxycarbonyl, paramethoxybenzyl, dialkylsilane, diaryldialkoxysilane, imine, orthoester, acetal, beta-thiopropionate, ketal, phosphoramidate, hydrazine, vinyl ether, aconityl, polyketal and trityl. The cleavable moiety may be selected from the group consisting of

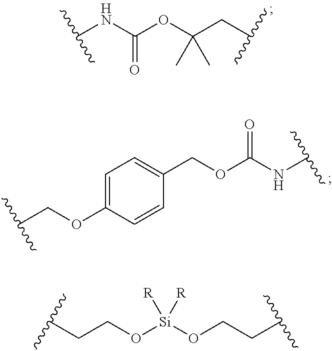

(where R is selected from methyl, ethyl, isopropyl, tert-butyl or phenyl);

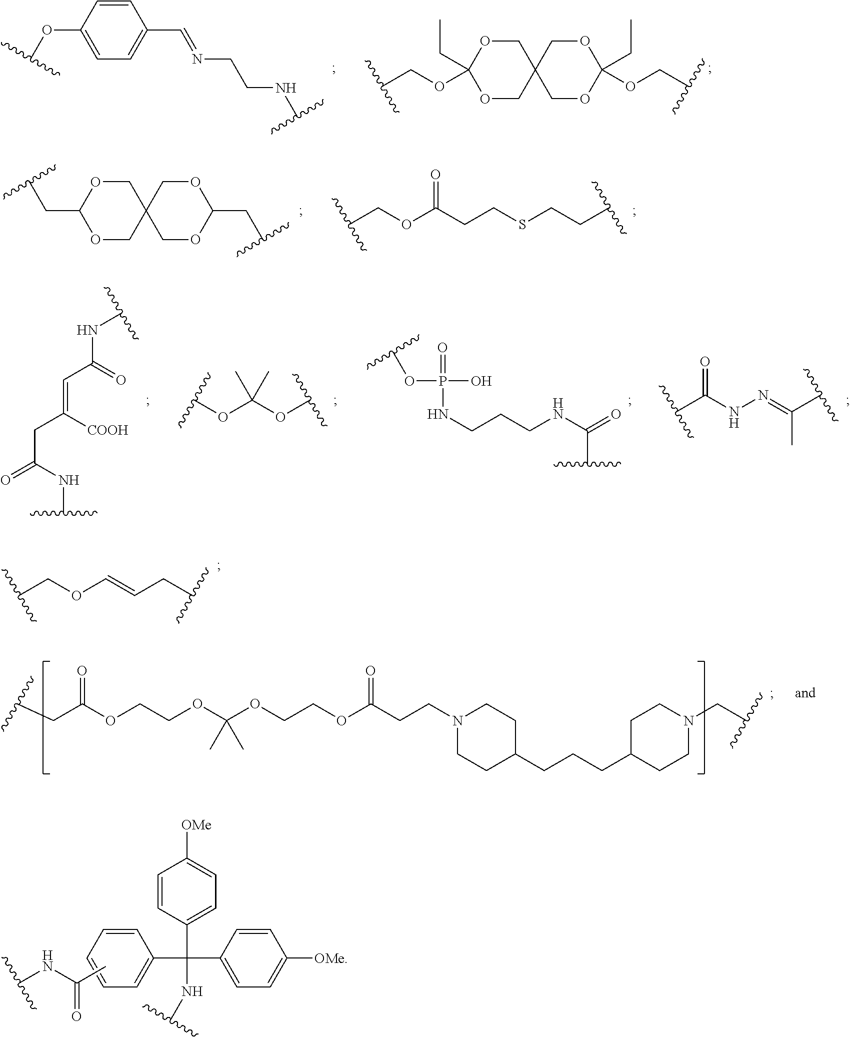

Exemplary acidic reagents include, but are not limited to, trifluoroacetic acid or formic acid.

The linker may comprise two or more moieties that are linked to each other by a cross-linker moiety. The cross-linker moiety may be at least one of alkyl groups, amide groups or combinations thereof. The alkyl groups may contain 1 to 10 carbon atoms.

The compound may be selected from

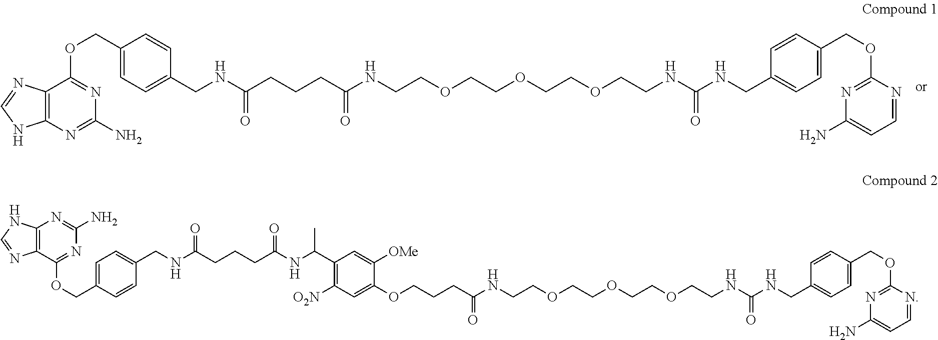 [SMILES: Nc1ccnc(OCc2ccc(CNC(=O)NCCOCCOCCOCCNC(=O)CCCC(=O)NCc3ccc(COc4nc(N)nc5[nH]cnc45)cc3)cc2)n1 ; "or" ; COc1cc(C(C)NC(=O)CCCC(=O)NCc2ccc(COc3nc(N)nc4[nH]cnc34)cc2)c([N+](=O)[O-])cc1OCCCC(=O)NCCOCCOCCOCCNC(=O)NCc1ccc(COc2nccc(N)n2)cc1]

For compound 1, the various groups of the compound are shown below:

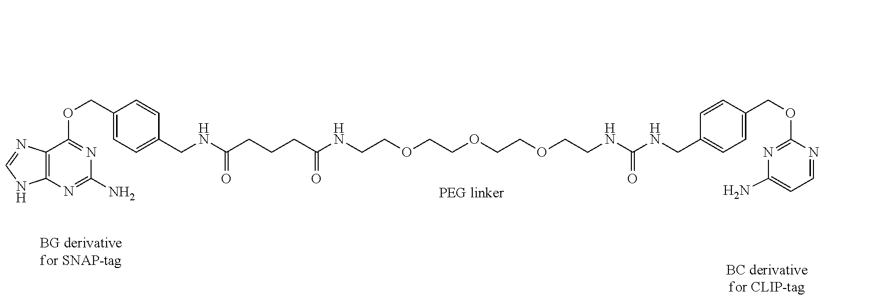 [SMILES: Nc1ccnc(OCc2ccc(CNC(=O)NCCOCCOCCOCCNC(=O)CCCC(=O)NCc3ccc(COc4nc(N)nc5[nH]cnc45)cc3)cc2)n1]

For compound 2, the various groups of the compound are shown below:

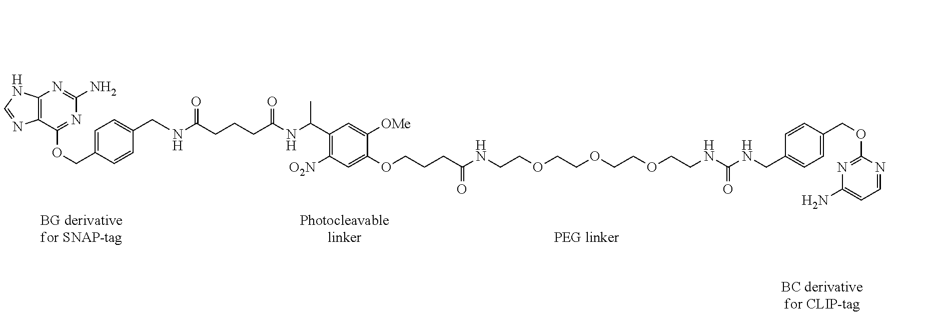 [SMILES: COc1cc(C(C)NC(=O)CCCC(=O)NCc2ccc(COc3nc(N)nc4[nH]cnc34)cc2)c([N+](=O)[O-])cc1OCCCC(=O)NCCOCCOCCOCCNC(=O)NCc1ccc(COc2nccc(N)n2)cc1]

The compound may be cell penetrable. The compound may be biologically inert and may not be a reactant or a target substrate in a biochemical reaction. The compound may allow for multiplex dimerization. The compound may be configured by adjusting the moieties in the linker portion to allow for optical or other types of selective control.

There is also provided an oligomer. The oligomer comprises a pair of ligands that are, coupled to respective proteins to form ligand/protein pairs, wherein each ligand/protein pair is individually coupled to a linker, and wherein each ligand is independently selected from the group consisting of a substituted benzylguanine derivative, a substituted benzylcytosine derivative, a haloalkyl moiety, a drug, a peptide, a hormone, an inorganic compound and a further protein.

Each of the ligand/protein pair may be independently selected from the group consisting of benzylguanine/SNAPtag, benzylcytosine/CLIPtag, rapamycin/FK506 binding protein (FKBP), doxycycline/Tetr and HA peptide/anti-HA scFV.

The oligomer may be selected from the group consisting of SNAPtag/benzylguanine-linker-benzylguanine/SNAPtag, SNAPtag/benzylguanine-linker-benzylcytosine/CLIPtag, CLIPtag/benzylcytosine-linker-benzylguanine/SNAPtag and CLIPtag/benzylcytosine-linker-benzylcytosine/CLIPtag.

The linker may comprise water-soluble polymeric moiety as mentioned above. The linker may further comprise one or more cleavable moiety as mentioned above.

There is also provided a method of dimerizing a pair of proteins. The method comprises the step of incubating the pair of proteins with a compound comprising at least two ligands that are individually coupled to a linker, wherein each ligand is independently selected from the group consisting of a substituted benzylguanine derivative, a substituted benzylcytosine derivative, a haloalkyl moiety, a drug, a hormone, an inorganic compound and a further protein, the ligands being respective substrates for the proteins of the protein pair.

The incubating step may be conducted in vivo or in vitro. The incubating step may comprise the step of selecting the concentration of the compound from a range of about 100 nM to about 50 μM, about 400 nM to about 50 μM, about 1 μM to about 50 μM, about 4 μM to about 50 μM, about 10 μM to about 50 μM, about 100 nM to about 400 nM, about 100 nM to about 1 μM, about 100 nM to about 4 μM, or about 100 nM to about 10 μM. The concentration of the compound when incubating with the protein pair may be about 100 nM, 400 nM, 1 μM, 4 μM or 10 μM.

The protein of the protein pair may be incorporated into a plasmid and transfected into a cell of interest.

There is also provided a method of altering a biological function in a cell, comprising the step of forming a dimer by incubating a pair of proteins with a compound comprising at least two ligands that are individually coupled to a linker comprising a cleavable moiety, wherein each ligand is independently selected from the group consisting of a substituted benzylguanine derivative, a substituted benzylcytosine derivative, a haloalkyl moiety, a drug, a hormone, an inorganic compound and a further protein, the ligands being respective substrates for the proteins of the protein pair.

The step of forming the dimer may cause the biological function to occur or the step of forming the dimer may inhibit the biological function.

The method may further comprise the step of cleaving the linker to thereby stop the progression of the biological function or in the alternative, the step of cleaving the linker may promote the occurrence of the biological function.

There is also provided a method of forming the compound, comprising the step of reacting a first ligand with a second ligand, wherein'each ligand is independently selected from the group consisting of a substituted benzylguanine derivative, a substituted benzylcytosine derivative, a haloalkyl moiety, a drug, a hormone, an inorganic compound and a protein.

The first ligand or second ligand may be linked to a water-soluble polymeric moiety as mentioned above. The first ligand or second ligand may be further linked to a cleavable moiety as mentioned above.

In order to form the compound, one of the ligands may have an amine terminal group that can react with a carboxylic functional group on the other ligand to form an amide bond. For example, the carboxylic functional group may be a succinimidyl ester group.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows the ¹H-NMR spectrum and structural formula of Compound 1.

FIG. 2 shows the mass spectrum of Compound 1 along with the mass spectrum peak list.

FIG. 3 shows the ¹H-NMR spectrum and structural formula of Compound 2.

FIG. 4 shows the mass spectrum of Compound 2.

FIG. 5 is a schematic diagram showing the dimerization of SNAP-eGFP and CLIP-eGFP in the presence of Compound 1 and Compound 2.

FIG. 6 is a number of western blots against eGFP. FIG. 6a shows the formation of dimer in the presence of SNAP-eGFP and CLIP-eGFP. FIG. 6b shows the effect of increasing concentrations of compound 1 on dimer formation. FIG. 6c shows the effect of incubation time of compound 1 on dimer formation. FIG. 6d shows the effect of increasing concentrations of compound 2 and UV illumination on dimer formation.

FIG. 7 is a schematic diagram showing the mechanism behind the up-regulation of luciferase.

FIG. 8a is a graph showing the relative luciferase levels as a result of increasing concentrations of compound 1 and at an exemplified concentration of compound 2. FIG. 8b is a graph showing the relative luminescence as a result of different light sources used and when harvested at different time periods. FIG. 8c is similar to FIG. 8b but with different concentration of compound 2 added and illumination time. FIG. 8d is a graph showing the effect of DMSO on the relative luminescence, with and without UV illumination.

### EXAMPLES

Non-limiting examples of the invention will be further described in greater detail by reference to specific Examples, which should not be construed as in any way limiting the scope of the invention.

### Example 1

### Synthesis of Compound 1

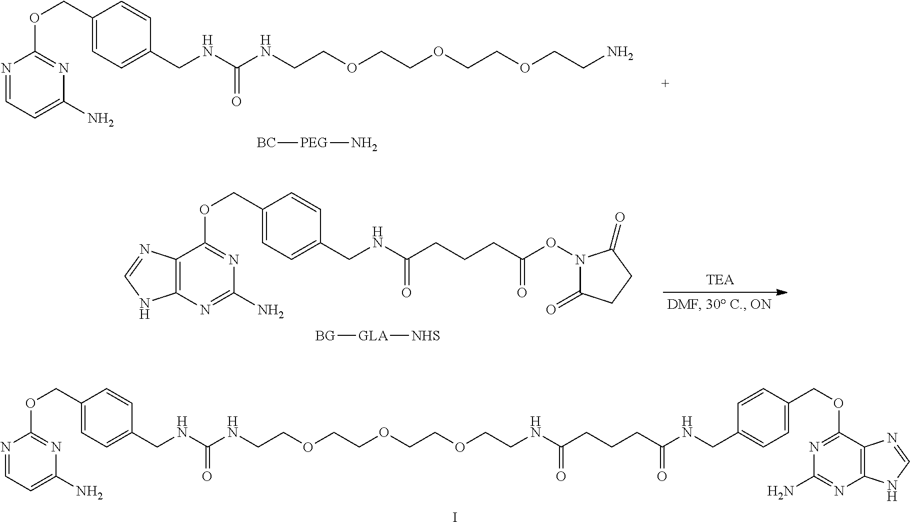

BC-PEG-NH₂ (1.86 mg, 4.15 μmol, obtained from New England Biolabs of Singapore) was dissolved in 400 μL of anhydrous dimethylformamide (DMF) and added to triethylamine (63 mg, 6.23 μmol). BG-GLA-NHS (2 mg, 4.15 μmol, obtained from New England Biolabs of Singapore) was dissolved in 400 μL of anhydrous DMF and added to the reaction mixture. The reaction mixture was stirred at 30° C. overnight. The solvent was removed under reduced pressure. The resultant reaction was dissolved in 1:1 acetonitrile/water (1 mL) and DMF (200 μL). The reagents were obtained commercially from Acros Chemicals (of New Jersey of the United States of America) as well as from Sigma-Aldrich (of Missouri of the United States of America) and used as is. The reaction was purified by high performance liquid chromatography. Compound 1 was obtained as a white powder (4.4 mg, quant) having the following properties.

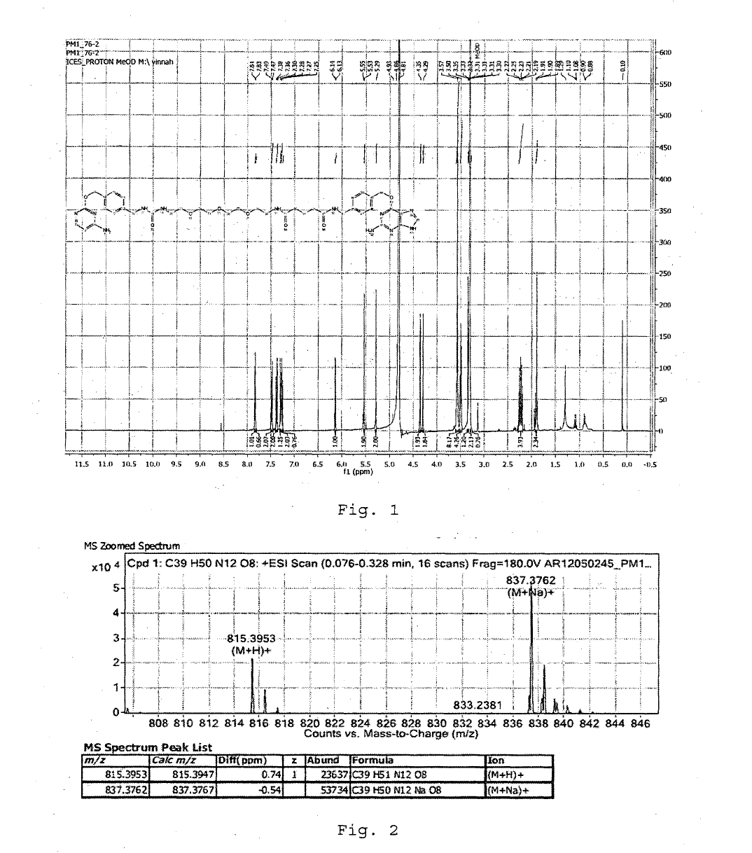

¹H NMR (400 MHz, methanol-d4) δ 1.88-1.92 (m, 2H), 2.23 (dt, J=15.0, 7.5 Hz, 4H), 3.29 (s, 1H), 3.33 (d, J=1.6 Hz, 2H), 3.35 (s, 1H), 3.47-3.53 (m, 4H), 3.54-3.61 (m, 8H), 4.29 (s, 2H), 4.35 (s, 2H), 5.29 (s, 2H), 5.53 (s, 2H), 6.13 (d, J=5.9 Hz, 1H), 7.25 (s, 1H), 7.27 (d, J=4.4 Hz, 2H), 7.30 (s, 1H), 7.37 (d, J=8.2 Hz, 2H), 7.48 (d, J=8.1 Hz, 2H), 7.83 (d, J=5.9 Hz, 2H). The ¹H-NMR spectrum of compound 1 is shown in FIG. 1.

HRMS (ESI+) m/z calc'd 814.39, found 815.3953 [M+H]⁺. The mass spectrum of compound 1 is found in FIG. 2.

### Example 2

### Synthesis of Compound 2

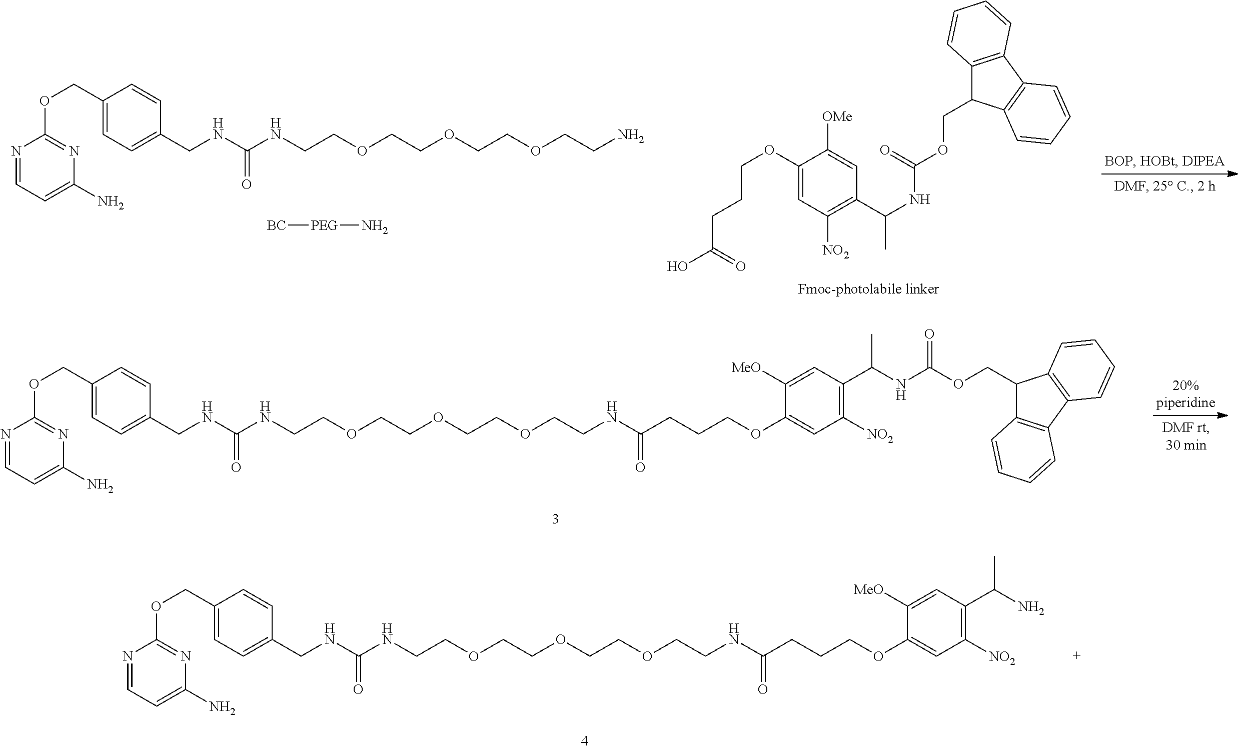

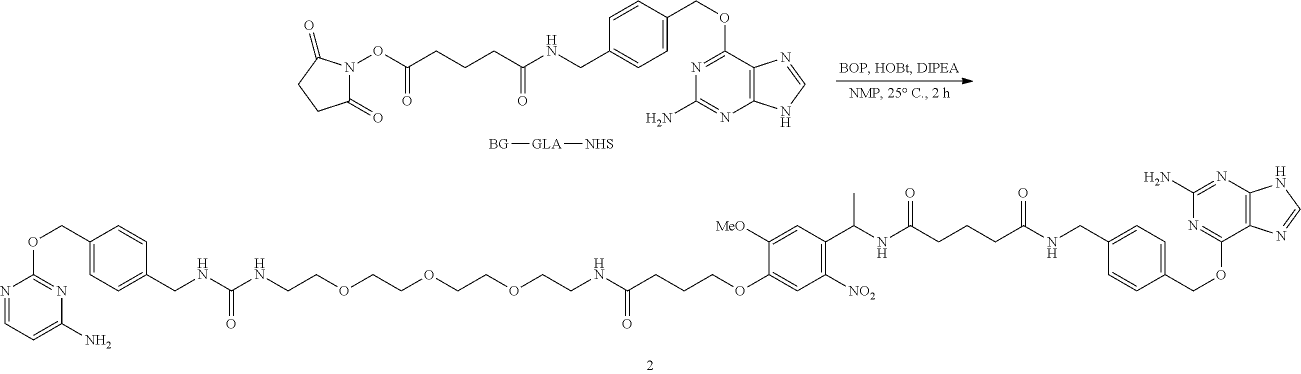

Fmoc-labile linker (4.6 mg, 8.92 umol, obtained from Advanced ChemTech of Kentucky of the United States of America), Benzotriazol-1-yloxy)tris(dimethylamino)-phosphonium hexafluorophosphate (BOP) (3.9 mg, 8.92 umol) and Hydroxy-benzotriazole (HOBt) (1.2 mg, 8.92 umol) were dissolved in DMF (100 uL) each respectively and added to BC-PEG-NH₂ (2 mg, 4.46 umol) dissolved in DMF (100 uL). N,N-Diisopropylethylamine (DIPEA) (2.31 mg, 17.8 umol) was added to the reaction mixture and stirred for 2 hours. The reagents were obtained commercially from Acros Chemicals (of New Jersey of the United States of America) as well as from Sigma-Aldrich (of Missouri of the United States of America) and used as is. After stirring, the reaction mixture was filtered and purified by reversed phase HPLC (RP-HPLC) to obtain compound 3 (2.2 mg, 51.8%) m/z: calc'd 951.42, found 951.3 (M+H).

Compound 3 (2.2 mg, 2.3 umol) was dissolved in DMF (360 uL). Piperidine (90 uL) was added and stirred for 30 minutes. After stirring, the reaction mixture was filtered and purified by RP-HPLC to obtain compound 4 (1.7 mg, Quant.) m/z: calc'd 729.35, found 729.56 (M+H).

Compound 4 (1.7 mg, 2.33 umol), BOP (2.1 mg, 4.66 umol) and HOBt (0.6 mg, 4.66 umol) were dissolved in DMF (100 uL) respectively and added to BG-GLA-NHS (2 mg, 4.15 umol) dissolved in DMF (100 uL). DIPEA (1.2 mg, 9.33 umol) was added to the reaction mixture and stirred for 4 hours. After stirring, the reaction mixture was filtered and purified by RP-HPLC to obtain the product, Compound 2 (0.7 mg, 27.4% having the following properties.

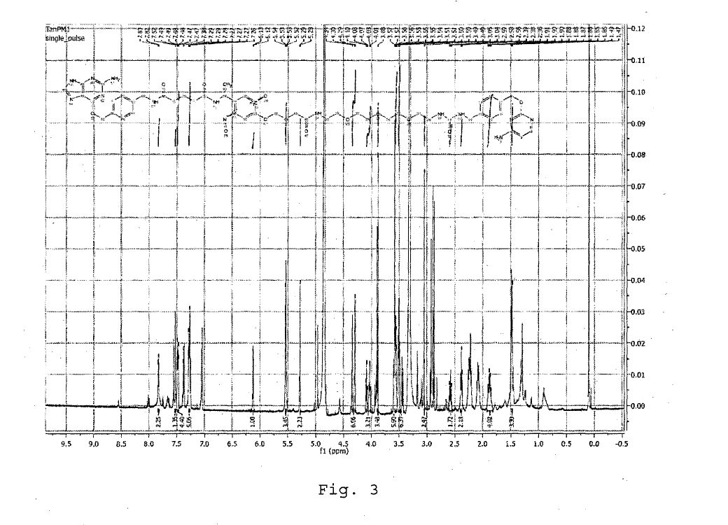

¹H NMR (500 MHz, MeOD) δ 1.47 (d, 3H), 1.81-1.94 (m, 4H), 2.39 (t, J=7.2 Hz, 3H), 2.52-2.62 (t, 2H), 3.04 (m, 2H), 3.50 (m, 6H), 3.51-3.60 (m, 6H), 3.88 (s, 3H), 4.32 (m, 6H), 5.28 (d, J=5.8 Hz, 3H), 5.50-5.57 (m, 3H), 6.12 (d, 1H), 7.23-7.32 (m, 5H), 7.43-7.52 (m, 4H), 7.52 (s, 1H), 7.83 (d, J=6.2 Hz, 2H). The ¹H-NMR spectrum of compound 2 is shown in FIG. 3.

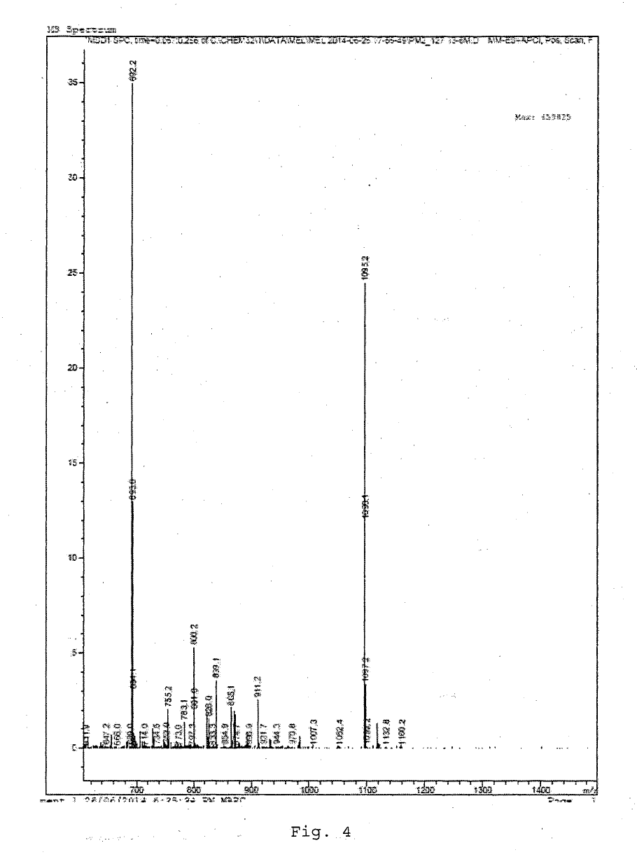

m/z: calc'd 1095.49, found 1095.2 (M+H). The mass spectrum of compound 2 is found in FIG. 4.

### Example 3

### Dimerization of SNAP-CLIP

### Preparation of Plasmids

Mammalian expression vectors for SNAP-eGFP and CLIP-eGFP were constructed from pSNAP_{f} and pCLIP_{f} (obtained from New England Biolabs of Singapore). eGFP was amplified using primers XhoI eGFP F and NotI eGFP R from pEGFP-NAD. The amplified DNA fragment was digested using XhoI/NotI and cloned into pSNAP_{f} and pCLIP_{f} for C-terminal fused eGFP-SNAP and eGFP-CLIP constructs respectively.

TABLE
List of Primers Used and Sequences

| Primer | Sequence |
|---|---|
| XhoI eGFP F | CGGATCCGCGTTTAAACTCGA |
| | GATGGTGAGCAAGGGCGAGGA |
| | GCTGTTCA |
| NotI eGFP R | TGGATCAGTTATCTATGCGGC |
| | CGCTCATTACTTCTTGTACAG |
| | CTCGTCCATGCCGAGA |
| NheI NLS CLIP | AAAAAAgctagcgctaccggt |
| | cgccaccatgatgcctgctgc |
| | caagagggtca |
| SNAP XhoI C | TTTTTTctcgagACCCAGCCC |
| | AGGCTTGCCCA |
| SNAP-p65 AD- | ACAGACTGGGCAAGCCTGGGC |
| HSF1AD N | TGGGTactagaagtgagccca |
| | tggaatttca |
| p65 AD-HSF1AD | GGATCCctagtggtggtggtg |
| histag BamHI | gtggtgggagacagtggggtc |
| | cttgg |
| SNAP NheI N | GCTAGCGATATCGGCGCGCCA |
| SNAP-gal4 N | ACAGACTGGGCAAGCCTGGGC |
| | TGGGTTCTTCTATCGAACAAG |
| | CATGCGATATTT |
| gal4 histag | ttttttGGATCCctagtggtg |
| BamHI | gtggtggtggtgCGATACAGT |
| | CAACTGTCTTTGACCTTT |

pGL4.35 (obtained from Promega of Singapore) contains firefly luciferase under the control of 9 UAS elements. SNAP was amplified from pSNAPf with SNAP-XhoI-C and SNAP NheI N; CLIP from pCLIPf with NheI NLC CLIP and SNAP-XhoI-C; Gal4 from pCMV-Gal4 with Gal4 HisTag BamHI and SNAP-Gal4 N; activation domains (AD2) from pHet-Act2-1 with SNAP-p65AD-HSF1AD N and p65 AD-HSF1AD HisTag BamHI. The individual PCR reactions were combined by SOEing PCR (Splicing by Overlap Extension) to make SNAP-Gal4 and CLIP-AD2 PCR products. These were then cloned into peGFP-C1 with the NheI and BamHI sites, removing eGFP in the process. Both constructs contained the nuclear localizing signal.

### Cell Culture

HEK293 cells were obtained from ATCC and grown in DMEM medium supplemented by 10% FCS and penicillin-streptomycin at 37° C. and 5% carbon dioxide. All experiments performed were carried out in 24 well plates seeded with 1×10⁵ cells per well. Transfection was performed with Lipofectamine 2000 (Invitrogen™, Life Technologies of Singapore) as per manufacturer's instructions the day after seeding. Medium was changed the day after transfection.

Treatment with Dimerizers

The dimerizers (or Compounds 1 and 2) were dissolved to 1 mM in DMSO and diluted further with DMSO so all applications were done by adding 4 μl of DMSO with compound 1 dissolved in it to 400 μl of medium for each well. Compound 1 was added at the mentioned concentration two days after transfection and incubated for 3 to 6 hours before the cells were harvested with RIPA Lysis and Extraction buffer (from Thermo Fisher Scientific Inc of Illinois of the United States of America). Compound 2 was added at the mentioned concentration three days after transfection and incubated for a specified duration before the cells were harvested.

FIG. 5 is a schematic diagram showing the dimerization of SNAP-eGFP and CLIP-eGFP in the presence of Compound 1 and Compound 2.

### Compound 1 Induced Dimerization of SNAP-eGFP and CLIP-eGFP Specifically

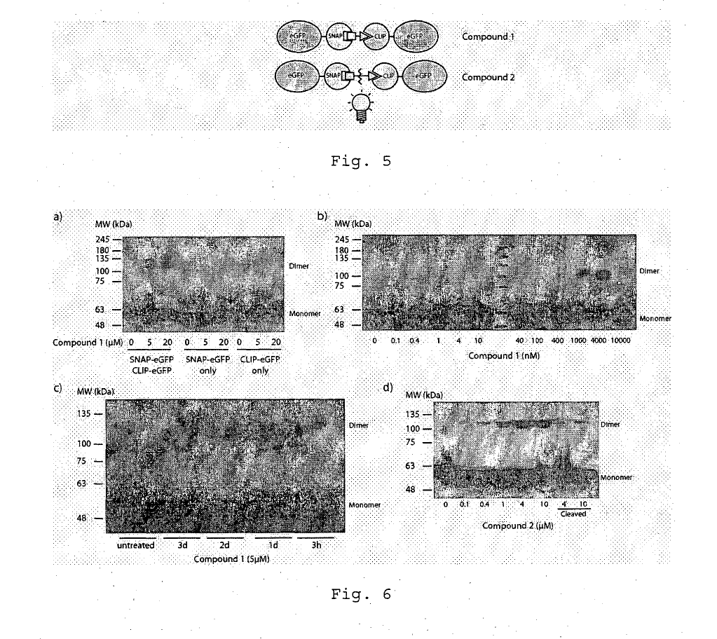

HEK293 cells were transfected with 500 ng of SNAP-eGFP and CLIP-eGFP together or 1 μg of SNAP-eGFP or CLIP-eGFP only. 2 days after transfection, 0, 5 or 20 μM of Compound 1 was applied to the cells with fresh medium and cells were harvested 6 hours after. FIG. 6a is a western blot showing the dimerized eGFP only when SNAP-eGFP and CLIP-eGFP were present in the cells at the various concentrations of Compound 1, while no dimerization occurred with SNAP-eGFP or CLIP-eGFP only, suggesting that heterodimerization occurred specifically and that compound 1 could penetrate cells in culture when applied with DMSO.

Heterodimerization also proceeded in a dose-dependent manner with dimerization occurring only when 400 nM of 1 was applied, as shown in FIG. 6b. Here, HEK cells were transfected with 500 ng of SNAP-eGFP and CLIP-eGFP plasmids each. Increasing amounts of compound 1 (0.1 nM, 0.4 nM, 1 nM, 4 nM, 10 nM, 40 nM, 100 nM, 400 nM, 1000 nM, 4000 nM and 10,000 nM) were applied 2 days after transfection and the cells were harvested 3 hours after.

Maximal Dimerization is Achieved within 24 Hours of Addition of Compound 1 and Resultant Dimer is Stable for at least Three Days in the Cell

HEK293 cells transfected with 250 ng of SNAP-eGFP and CLIP-eGFP were treated 3 days, 2 days, 1 day or 6 hours prior to harvesting with 5 μM of compound 1, in duplicate. FIG. 6c is a western blot showing that dimerization is near saturation 3 hours after application. 24 hours after application, maximal dimerization was achieved with no change (no increase or decrease) in dimerization after 3 days of treatment, suggesting that the dimer acted rapidly (within 24 hours) and was stable when complexed in the SNAP-CLIP dimer.

Photocleavable Dimerizer (Compound 2) Induced Dimerization of SNAP-eGFP and CLIP-eGFP Specifically and Reversibly when Illuminated with 365 nm Light

HEK293 cells were transfected with 250 ng of SNAP-eGFP and CLIP-eGFP. 3 days after transfection, increasing concentrations of 0.1 to 10 μM (0.1 μM, 0.4 μM, 1 μM, 4 μM, and 10 μM) of photocleavable dimerizer (Compound 2) were applied to the cells with fresh medium and the cells were harvested 6 hours after. FIG. 6d is a western blot showing that the photocleavable dimerizer, compound 2, could induce dimerization at lower concentrations than compound 1 and that 365 nm illumination for 10 minutes was capable of partially reversing dimerization. The cleaved samples were equivalent volumes of uncleaved samples subjected to 365 nm illumination for 10 minutes.

Both Dimerizers are Capable of Upregulation of Gene Expression through Dimerization of a DNA Binding Domain and a Transcriptional Activator

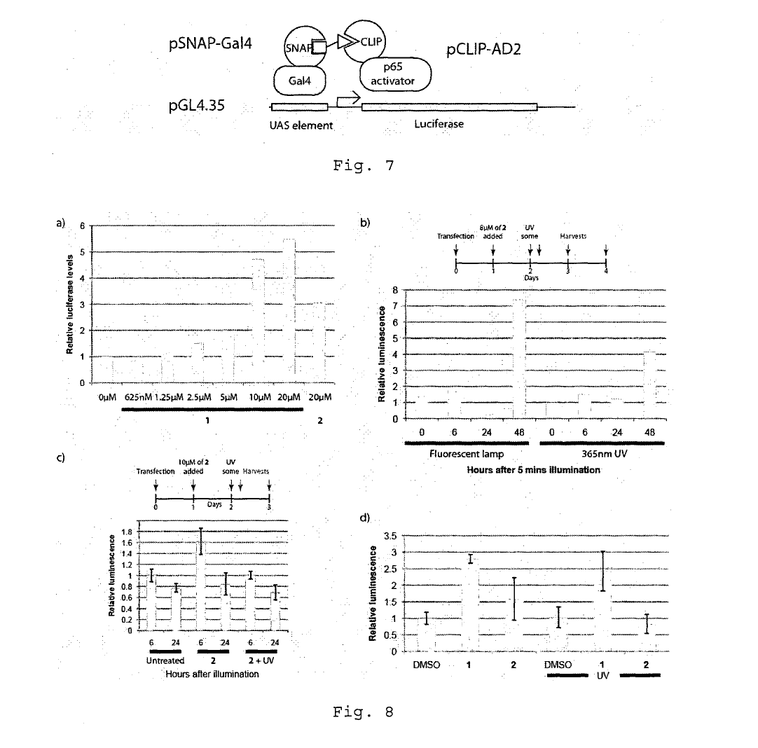

HEK293 cells (in a 24-well plate) transfected with 400 ng pSNAP-Gal4 (Gal4 is a DNA-binding domain fused to SNAP), 200 ng pCLIP-AD2 (AD2 is a transcriptional activator fused to CLIP) and 100 ng pGL4.35 (GL4.35 is a luciferase reporter construct containing nine Gal4 binding motif for gene activation) were treated with stated amounts of either Compound 1 or 2 in 400 μl medium 1 day after transfection. Cells were harvested 24 hours after treatment and the levels of luciferase measured. FIG. 7 is a schematic diagram showing the up-regulation of luciferase.

FIG. 8a shows that luciferase was produced in a dose-dependent fashion with increasing concentration of compound 1. FIG. 8a also shows that luciferase was produced in the presence of compound 2. This demonstrates the ability of the dimerizers to control gene expression in cell culture. Moreover, exposure to UV illumination renders compound 2 less effective (see FIG. 8b to FIG. 8d) at luciferase upregulation, demonstrating that the gene activation is reversible by UV irradiation without the need to change media.

### Effect of Light Sources on Luciferase Up-Regulation

As shown in FIG. 8b, subjecting the cells to different types of light source affected the up-regulation of luciferase. Here, the cells were transfected as above and 8 μM of compound 2 was added to the cells one day after transfection. One day after, the cells were either illuminated with a fluorescent lamp or with a 365 nm UV lamp for 5 minutes. 0, 6, 24 or 48 hours later, the cells were harvested and the levels of luciferase measured. FIG. 8b shows that UV illumination, which cleaved compound 2, resulted in lower expression of luciferase as compared to illumination using a fluorescent lamp.

### Effect of Increased Concentration and UV Illumination

The cells were transfected as above and 10 μM of compound 2 was added to the cells one day after transfection. One day after, the cells were illuminated with a 365 nm UV lamp for 10 minutes. As shown in FIG. 8c, 6 or 24 hours later, the cells were harvested and the levels of luciferase measured. Experiments were carried out in triplicates. The purpose of this experiment is to use a slightly higher amount of compound 2 and a longer UV irradiation time to optimize the linker cleavage.

### Effect of DMSO

HEK293 cells were transfected with the same ratio of plasmids in a 96-well plate and treated with 2% DMSO, 20 μM compound 1 or 20 μM compound 2 one day after transfection. Just after application, some of the cells were illuminated with 365 nm UV for 10 minutes. One day after, the cells were harvested. As shown in FIG. 8d, it is notable that cells with compound 2 showed decreased luciferase expression after UV illumination while cells with compound 1 did not, demonstrating the ability of the UV light to break the transcription dimer. The use of DMSO here (which is present as a solvent in low concentrations for dissolving the compound) at a higher concentration of 2% was to demonstrate that the presence of DMSO did not affect luciferase expression.

### Applications

The compound may facilitate the dimerization of naturally non-interacting proteins in a cell. The compound may induce specific dimerization. Hence, the compound may be used to control protein activity in a cell. The compound may control gene expression via gene switches in a cell.

The compound may be used to control biochemical processes that are either activated or inhibited by heterodimerization, such as transcription, receptor activation or protein degradation in a cell. The compound may aid in activating transcription by bringing a DNA recognition domain together with a transcriptional activator. The compound may aid in coupling two proteins together for a specific function in vitro, such as heterodimerization-induced enzymatic activity.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1-45. (canceled)

46. A compound comprising at least two ligands that are individually coupled to a linker, wherein each ligand is independently selected from the group consisting of a substituted benzylguanine derivative, a substituted benzylcytosine derivative, a haloalkyl moiety, a drug, a peptide, a hormone, an inorganic compound and a protein,
optionally wherein said linker comprises a water-soluble polymeric moiety,
optionally wherein said water-soluble polymeric moiety comprises monomers selected from the group consisting of alkylene glycols, alkylene pyrrolidones, alkylene alcohols, carboxylic acids, alkylene amides, alkyl acetates, hydroxyalkyls, oxazolines, phosphates, phosphazenes, saccharides, peptides, and combinations thereof,
optionally wherein said water-soluble polymeric moiety is selected from the group consisting of polyethylene glycol, polyvinyl pyrrolidone, polyvinyl alcohol, polyacrylic acid, polyacrylamides, N-(2-hydroxypropyl)methacrylamide, divinyl ether-maleic anhydride, polyoxazoline, polyphosphate, polyphosphazne, xanthan gum, pectin, chitin, chitosan, dextran, carrageenan, guar gum, hydroxypropylmethyl cellulose, hydroxypropyl cellulose, hydroxyethyl cellulose, sodium carboxy methyl cellulose, hyaluronic acid, albumin, starch and copolymers thereof, and
optionally wherein said linker further comprises one or more cleavable moiety.

47. The compound of claim 46, wherein said cleavable moiety is cleavable by at least one of enzyme, basic reagent, reducing agent, photo-irradiation, acidic reagent and oxidizing agent, optionally
wherein said enzyme is selected from the group consisting of trypsin, thrombin, cathepsin B, cathepsin D, cathepsin K, caspase 1, matrix matelloproteinase, phosphodiesterase, phospholipidase, esterase and beta-galactosidase, optionally
wherein said cleavable moiety that is cleavable by a basic reagent is selected from the group consisting of dialkyl dialkoxysilane, cyanoethyl group, sulfone, ethylene, glycolyl disuccinate, 2-N-acyl nitrobenzenesulfonamide, alpha-thiophenylester, unsaturated vinyl sulfide, sulfonamide after activation, malondialdehyde-indole derivative, levulinoyl ester, hydrazine, acylhydrazone and alkyl thioester and optionally
wherein said cleavable moiety that is cleavable by a reducing agent is at least one of a disulfide-containing moiety and an azo compound.

48. The compound of claim 47, wherein said cleavable moiety that is cleavable by photo-irradiation is selected from the group consisting of 2-nitrobenzyl derivative, phenacyl ester, 8-quinolinyl benzenesulfonate, coumarin, phosphotriester, bis-arylhydrazone and bimane-bisthiopropionic acid and optionally
wherein said cleavable moiety that is cleavable by photo-irradiation is selected from the group consisting of 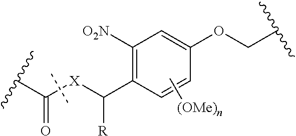
wherein X is —NH— or —O— and R is methyl or hydrogen;

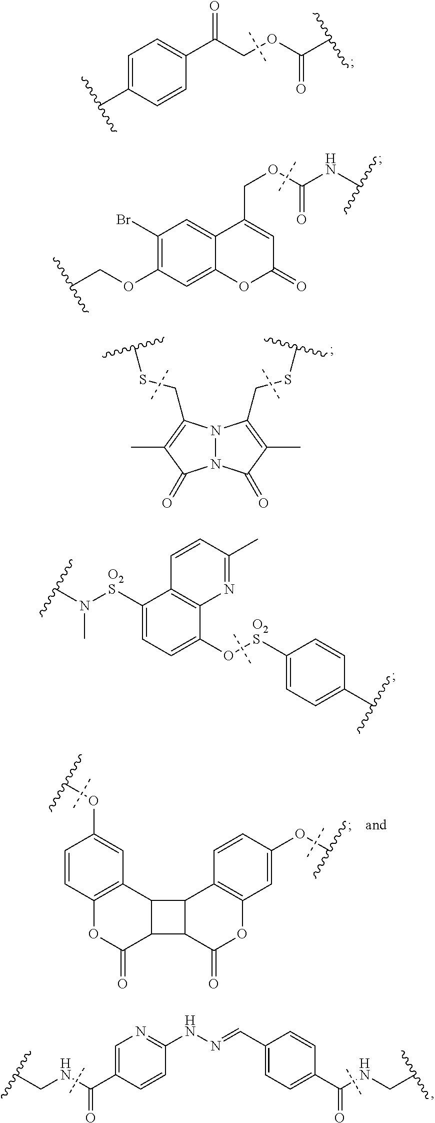

49. The compound of claim 47, wherein said cleavable moiety that is cleavable by an acidic reagent is selected from the group consisting of tert-butyloxycarbonyl, paramethoxybenzyl, dialkylsilane, diaryldialkoxysilane, imine, orthoester, acetal, beta-thiopropionate, ketal, phosphoramidate, hydrazine, vinyl ether, aconityl, polyketal and trityl and optionally
wherein said cleavable moiety that is cleavable by an acidic reagent is selected from the group consisting of 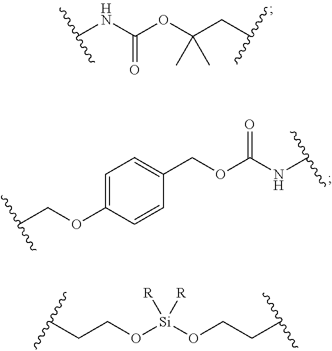
where R is selected from methyl, ethyl, isopropyl, tert-butyl or phenyl;

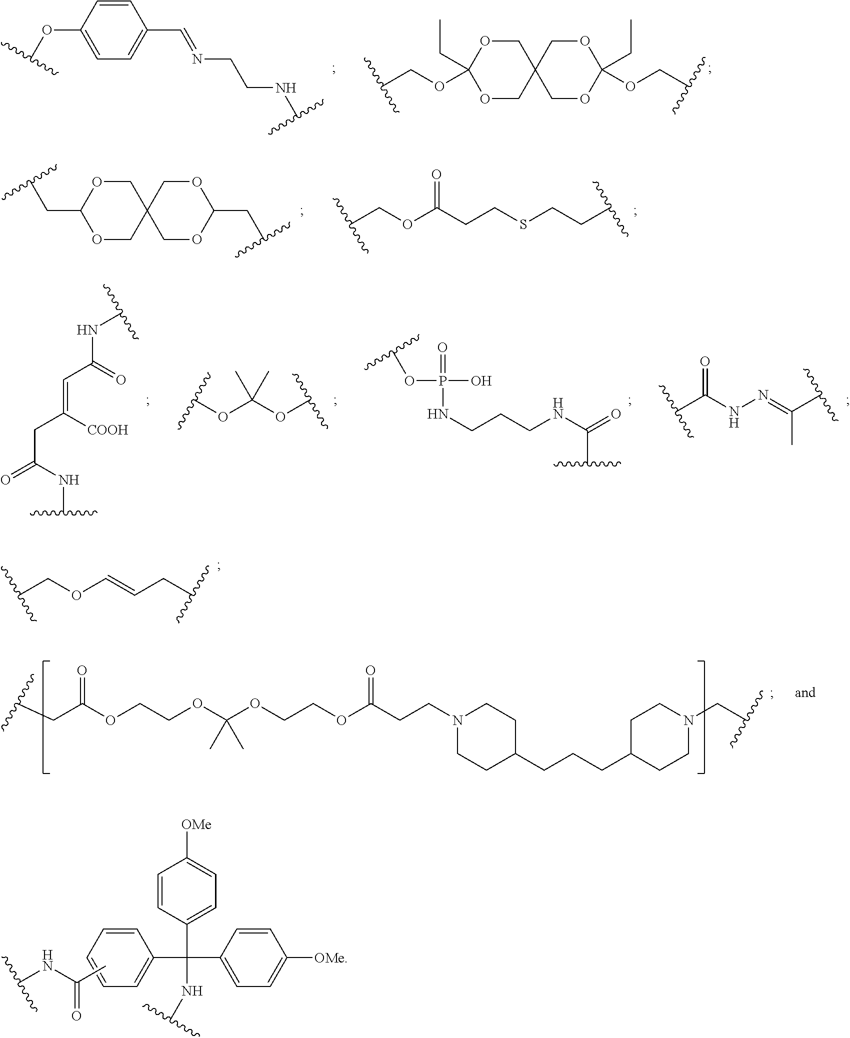

50. The compound of claim 46, wherein when said linker comprises two or more moieties, said moieties are linked to each other by a cross-linker moiety, optionally
wherein said cross-linker moiety is at least one of alkyl groups, amide groups or combinations thereof and optionally
wherein said alkyl groups contain 1 to 10 carbon atoms.

51. The compound of claim 46, wherein said ligands are both selected from a substituted benzylguanine derivative or alternatively
wherein said ligands are both selected from a substituted benzylcytosine derivative.

52. The compound of claim 46, wherein said substituted benzylguanine derivative has the following formula (I): 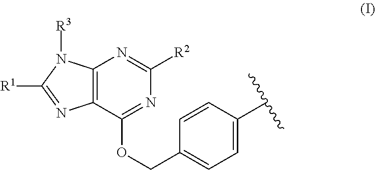
wherein
R¹ is selected from hydrogen or alkyl;
R² is selected from amino, hydroxyl, alkylamino, dialkylamino or acylamino;
R³ is selected from hydrogen, aminoalkyl, alkyl or dialkylamino; and [symbol]
denotes the point of attachment to said linker, optionally
wherein said R¹ and R³ are both hydrogen and R² is amino and optionally
wherein said alkyl, alkylamino, dialkylamino or acylamino independently contain 1 to 10 carbon atoms.

53. The compound of claim 46, wherein said substituted benzylcytosine derivative has the following formula (II): 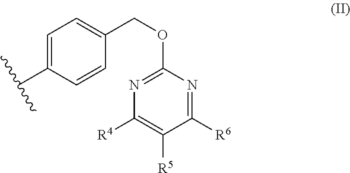
wherein
R⁴ is selected from amino, hydroxyl, alkylamino, dialkylamino or acylamino;
R⁵ is selected from hydrogen, aminoalkyl, alkyl or dialkylamino;
R⁶ is selected from hydrogen, aminoalkyl, alkyl or dialkylamino; and [symbol]
denotes the point of attachment to said linker, optionally.
wherein said R⁴ is amino and R⁵ and R⁶ are both hydrogen and optionally
wherein said alkyl, alkylamino, dialkylamino or acylamino independently contain 1 to 10 carbon atoms.

54. The compound of claim 46, wherein said compound is selected from

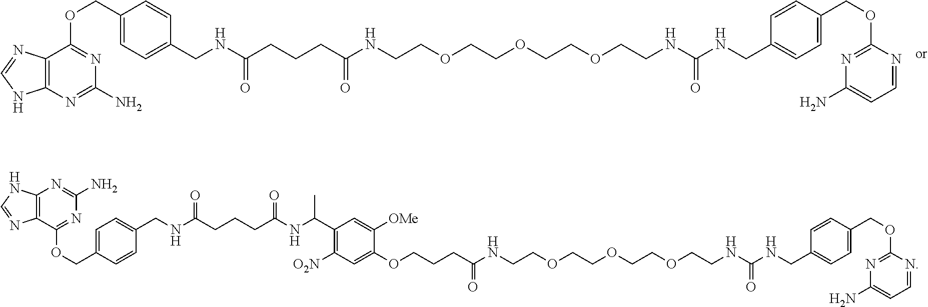 [SMILES: Nc1ccnc(OCc2ccc(CNC(=O)NCCOCCOCCOCCNC(=O)CCCC(=O)NCc3ccc(COc4nc(N)nc5[nH]cnc45)cc3)cc2)n1 ; "or" ; COc1cc(C(C)NC(=O)CCCC(=O)NCc2ccc(COc3nc(N)nc4[nH]cnc34)cc2)c([N+](=O)[O-])cc1OCCCC(=O)NCCOCCOCCOCCNC(=O)NCc1ccc(COc2nccc(N)n2)cc1]

55. The compound of claim 46, wherein said drug is selected from the group consisting of rapamycin, doxycycline and tetracycline.

56. The compound of claim 46, wherein said protein is a peptide tag and optionally
wherein said peptide tag is selected from the group consisting of a FLAG-tag, an AviTag, a calmodulin-tag, a HA-tag, a His-tag, a Myc-tag, a S-tag, a SBP-tag, a softag 1, a softag3, a V5 tag, a Xpress tag, an isopeptad, a SpyTag, glutathione-S-transferase-tag, green fluorescent protein-tag, maltose binding protein-tag, biotin carboxyl carrier protein-tag, Nus-tag, strep-tag, thioredoxin-tag, TC tag and Ty tag.

57. The compound of claim 46, wherein said inorganic compound is nickel.

58. An oligomer comprising a pair of ligands that are coupled to respective proteins to form ligand/protein pairs, wherein each ligand/protein pair is individually coupled to a linker, and wherein each ligand is independently selected from the group consisting of a substituted benzylguanine derivative, a substituted benzylcytosine derivative, a haloalkyl moiety, a drug, a peptide, a hormone, an inorganic compound and a further protein, optionally
wherein said ligand/protein pair is independently selected from the group consisting of benzylguanine/SNAPtag, benzylcytosine/CLIPtag, rapamycin/FK506 binding protein (FKBP), doxycycline/Tetr and HA peptide/anti-HA scFV and optionally
wherein said oligomer is selected from the group consisting of SNAPtag/benzylguanine-linker-benzylguanine/SNAPtag, SNAPtag/benzylguanine-linker-benzylcytosine/CLIPtag, CLIPtag/benzylcytosine-linker-benzylguanine/SNAPtag and CLIPtag/benzylcytosine-linker-benzylcytosine/CLIPtag.

59. The oligomer of claim 58, wherein said linker comprises a water-soluble polymeric moiety and optionally
wherein said linker further comprises one or more cleavable moiety.

60. A method of dimerizing a pair of proteins comprising the operation of incubating said pair of proteins with a compound comprising at least two ligands that are individually coupled to a linker, wherein each ligand is independently selected from the group consisting of a substituted benzylguanine derivative, a substituted benzylcytosine derivative, a haloalkyl moiety, a drug, a hormone, an inorganic compound and a further protein, said ligands being respective substrates for the proteins of said protein pair, optionally
wherein said incubating operation occurs in vivo or in vitro and optionally
wherein said incubating operation comprises the operation of selecting the concentration of said compound from the range of 300 nM to 50 μM.

61. A method of altering a biological function in a cell, comprising the operation of forming a dimer by incubating a pair of proteins with a compound comprising at least two ligands that are individually coupled to a linker comprising a cleavable moiety, wherein each ligand is independently selected from the group consisting of a substituted benzylguanine derivative, a substituted benzylcytosine derivative, a haloalkyl moiety, a drug, a hormone, an inorganic compound and a further protein, said ligands being respective substrates for the proteins of said protein pair.

62. The method of claim 61, wherein the operation of forming said dimer causes said biological function to occur.

63. The method of claim 62, further comprising the operation of cleaving said linker to thereby stop the progression of said biological function.

64. The method of claim 61, wherein the operation of forming said dimer inhibits said biological function, optionally
further comprising the operation of cleaving said linker to thereby promote the occurrence of said biological function.

65. A method of forming a compound comprising at least two ligands that are individually coupled to a linker, wherein each ligand is independently selected from the group consisting of a substituted benzylguanine derivative, a substituted benzylcytosine derivative, a haloalkyl moiety, a drug, a peptide, a hormone, an inorganic compound and a protein,
optionally wherein said linker comprises a water-soluble polymeric moiety,
optionally wherein said water-soluble polymeric moiety comprises monomers selected from the group consisting of alkylene glycols, alkylene pyrrolidones, alkylene alcohols, carboxylic acids, alkylene amides, alkyl acetates, hydroxyalkyls, oxazolines, phosphates, phosphazenes, saccharides, peptides, and combinations thereof,
optionally wherein said water-soluble polymeric moiety is selected from the group consisting of polyethylene glycol, polyvinyl pyrrolidone, polyvinyl alcohol, polyacrylic acid, polyacrylamides, N-(2-hydroxypropyl)methacrylamide, divinyl ether-maleic anhydride, polyoxazoline, polyphosphate, polyphosphazne, xanthan gum, pectin, chitin, chitosan, dextran, carrageenan, guar gum, hydroxypropylmethyl cellulose, hydroxypropyl cellulose, hydroxyethyl cellulose, sodium carboxy methyl cellulose, hyaluronic acid, albumin, starch and copolymers thereof, and
optionally wherein said linker further comprises one or more cleavable moiety, comprising the operation of reacting a first ligand with a second ligand, wherein each ligand is independently selected from the group consisting of a substituted benzylguanine derivative, a substituted benzylcytosine derivative, a haloalkyl moiety, a drug, a hormone, an inorganic compound and a protein, optionally
wherein said first ligand or said second ligand is linked to a water-soluble polymeric moiety and optionally
wherein said method comprises the operation of reacting said first ligand or said second ligand with a linker having a cleavable moiety.


SEQUENCE LISTING (9 sequences)

SEQ ID NO: 1 - DNA, 50 bp, Artificial Sequence
  primer
cggatccgcgtttaaactcgagatggtgagcaagggcgaggagctgttca

SEQ ID NO: 2 - DNA, 58 bp, Artificial Sequence
  primer
tggatcagttatctatgcggccgctcattacttcttgtacagctcgtccatgccgaga

SEQ ID NO: 3 - DNA, 53 bp, Artificial Sequence
  primer
aaaaaagctagcgctaccggtcgccaccatgatgcctgctgccaagagggtca

SEQ ID NO: 4 - DNA, 32 bp, Artificial Sequence
  primer
ttttttctcgagacccagcccaggcttgccca

SEQ ID NO: 5 - DNA, 52 bp, Artificial Sequence
  primer
acagactgggcaagcctgggctgggtactagaagtgagcccatggaatttca

SEQ ID NO: 6 - DNA, 47 bp, Artificial Sequence
  primer
ggatccctagtggtggtggtggtggtgggagacagtggggtccttgg

SEQ ID NO: 7 - DNA, 21 bp, Artificial Sequence
  primer
gctagcgatatcggcgcgcca

SEQ ID NO: 8 - DNA, 54 bp, Artificial Sequence
  primer
acagactgggcaagcctgggctgggttcttctatcgaacaagcatgcgatattt

SEQ ID NO: 9 - DNA, 60 bp, Artificial Sequence
  primer
ttttttggatccctagtggtggtggtggtggtgcgatacagtcaactgtctttgaccttt